(19) United States
(12) Patent Application Publication
(10) Pub. No.: US 2014/0029833 A1
(43) Pub. Date: Jan. 30, 2014

(54) X-RAY IMAGING APPARATUS, METHOD OF CONTROLLING X-RAY IMAGING APPARATUS, AND STORAGE MEDIUM

(71) Applicant: CANON KABUSHIKI KAISHA, Tokyo (JP)

(72) Inventor: Manami Hikosaka, Yokohama-shi (JP)

(21) Appl. No.: 13/938,599

(22) Filed: Jul. 10, 2013

(30) Foreign Application Priority Data
Jul. 25, 2012 (JP) 2012-165138

(51) Int. Cl. A61B 5/00

(52) U.S. Cl. CPC A61B 5/0033
USPC 382/132

(57) ABSTRACT

An X-ray imaging apparatus comprises an examination information input unit configured to input examination information necessary for an examination; an examination dividing operation reception unit configured to receive an instruction to divide the examination into a first examination associated with the examination information and a second examination unassociated with the examination information, during an interval between a start of the examination and an end of the examination; and an image distribution instruction reception unit configured to receive an instruction to distribute images which have been captured in the examination in association with one of the first examination and the second examination.

### BACKGROUND OF THE INVENTION

1. Field of the Invention

The present invention relates to an X-ray imaging apparatus, a method of controlling the X-ray imaging apparatus, and a storage medium.

2. Description of the Related Art

Conventionally, a medical image such as a captured X-ray image is sent to an image processing apparatus to be subjected to predetermined image processing. The apparatus then outputs the resultant image to an external apparatus such as an image display apparatus (viewer) or image archiving apparatus (server). At this time, the image processing apparatus also outputs additional information to be added to the captured X-ray image. Such additional information includes patient information such as a patient name, patient ID, birth date, and sex, and examination information such as an examination ID, examination comment, and examination date (see Japanese Patent Laid-Open No. 2004-290224).

According to the technique disclosed in Japanese Patent Laid-Open No. 2004-290224, when noticing that the operator has mistaken a given patient for an examination target, he/she may unintentionally change additional information of an image other than a change target during the same examination and diagnose an image of a patient other than the intended patient at the time of diagnosis. Performing correcting operation during an examination will increase the number of operation steps. This increases the risk of erroneous correction and prolongs the wait time for the patient.

### SUMMARY OF THE INVENTION

In consideration of the above problem, the present invention provides a technique of improving the operator's operability and reducing errors by decreasing the number of operation steps necessary for information correction when the operator notices that he/she has mixed up between patients during an examination.

According to one aspect of the present invention, there is provided an X-ray imaging apparatus comprising: an examination information input unit configured to input examination information necessary for an examination; an examination dividing operation reception unit configured to receive an instruction to divide the examination into a first examination associated with the examination information and a second examination unassociated with the examination information, during an interval between a start of the examination and an end of the examination; and an image distribution instruction reception unit configured to receive an instruction to distribute images which have been captured in the examination in association with one of the first examination and the second examination.

According to another aspect of the present invention, there is provided an X-ray imaging apparatus configured to manage a plurality of radiographic images in association with each examination, the apparatus comprising: a display control unit configured to cause a display unit to display a display screen for execution of the first examination in accordance with an instruction to start the first examination; and a storage unit configured to store information of the started examination in association with a radiographic image obtained by imaging, wherein the display control unit further causes the display unit to display, in the display screen for execution of the first examination, a GUI for receiving operation of associating, instead of the information of the first examination, information of a second examination which is different from the information of the first examination with at least one of a plurality of radiographic images with which the information of the first examination is associated.

Further features of the present invention will be apparent from the following description of exemplary embodiments with reference to the attached drawings.

### BRIEF DESCRIPTION OF THE DRAWINGS

FIG. 1 is a block diagram showing the arrangement of an X-ray imaging system according to the first embodiment;

FIG. 2 is a flowchart showing a processing procedure for an examination according to the first embodiment;

FIG. 3 is a flowchart showing a procedure for examination dividing processing for an examination under execution according to the first embodiment;

FIG. 4 is a view showing an example of an imaging screen GUI according to the first embodiment;

FIG. 5 is a view showing an example of a dividing operation execution confirmation dialog according to the first embodiment;

FIG. 6 is a view showing an example of an examination information re-input dialog according to the first embodiment;

FIG. 7 is a view showing an example of an imaging screen GUI after dividing according to the first embodiment;

FIG. 8 is a view showing an example of an imaging screen GUI after dividing according to the second embodiment; and

FIG. 9 is a view showing an example of a warning dialog upon a plurality of times of imaging of the same examination region according to the third embodiment.

### DESCRIPTION OF THE EMBODIMENTS

An exemplary embodiment(s) of the present invention will now be described in detail with reference to the drawings. It should be noted that the relative arrangement of the components, the numerical expressions and numerical values set forth in these embodiments do not limit the scope of the present invention unless it is specifically stated otherwise.

### First Embodiment

The first embodiment will exemplify an arrangement configured to receive an instruction to divide examination from the operator who has noticed that he/she has mixed up between patients during a series of examination procedures, in which a plurality of imaging procedures are performed, from the issuance of an instruction to start the examination to the issuance of an instruction to terminate the examination.

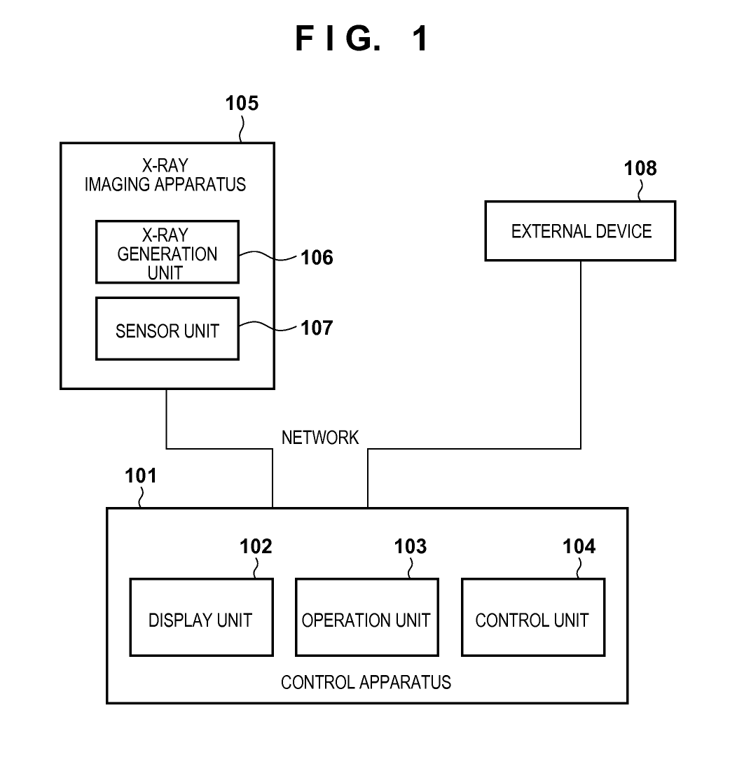

An example of the arrangement of an X-ray imaging system according to the first embodiment will be described with reference to FIG. 1. An X-ray imaging apparatus 105 includes an X-ray generation unit 106 and a sensor unit 107. The X-ray generation unit 106 irradiates an object with X-rays. The sensor unit 107 then forms the X-rays transmitted through the object into an image. The image captured by the X-ray imaging apparatus 105 is transmitted to a control apparatus 101 via a network.

The control apparatus 101 includes a display unit 102, an operation unit 103, and a control unit 104. The display unit 102 is a liquid crystal display or the like which displays the image data received from the X-ray imaging apparatus 105, a GUI (Graphical User Interface), and the like. The operation unit 103 includes a mouse, keyboard, and irradiation switch. The operation unit 103 receives command inputs of examination information such as patient information necessary for an examination from the operator or receives an X-ray imaging instruction. The operation unit 103 receives an examination start instruction, an examination termination instruction, and the like. The control unit 104 has a function of controlling the driving of the sensor unit 107 and X-ray generation unit 106 based on inputs from the operator, and a function of performing image processing for captured image data. The control unit 104 also manages correction data, X-ray imaging conditions, image data, and the like by using a database and the like.

The control unit 104 performs start processing/termination processing in accordance with an examination start instruction/termination instruction received from the operator, and performs editing processing for the captured image received from the X-ray imaging apparatus 105 and examination information. The control unit 104 also has a function of transferring image data to an external device 108 in response to an instruction from the operator which is received via the operation unit 103.

The external device 108 is an external device for saving and displaying the image data transferred from the control apparatus 101, and is more generally implemented as a PACS (Picture Archiving and Communication System).

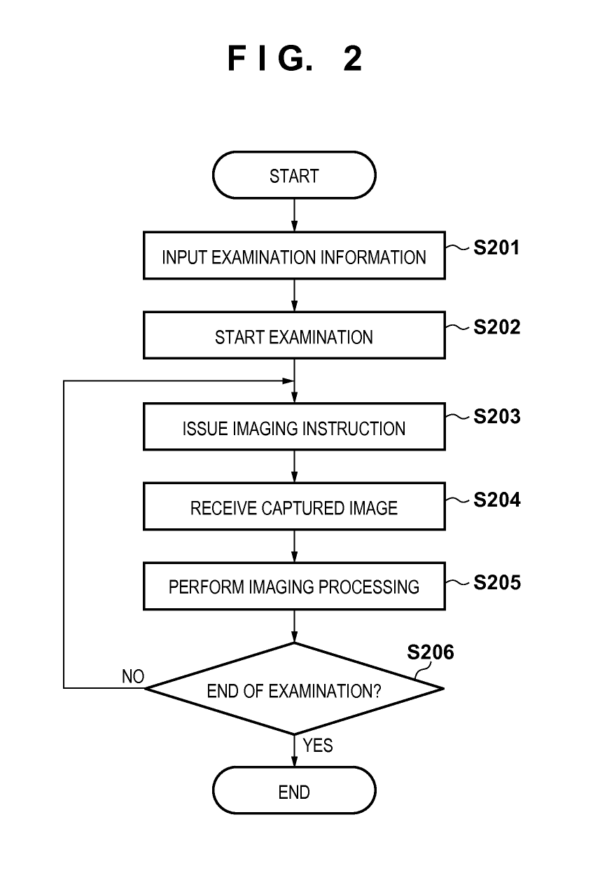

A general examination processing procedure will be described next with reference to the flowchart of FIG. 2.

In step S201, the operation unit 103 receives examination information inputs such as patient information from the operator. Note that the operator may input examination information by using the GUI displayed on the display unit 102. In step S202, the control apparatus 101 performs examination start processing in accordance with an examination start instruction from the operator.

In step S203, the operation unit 103 receives an imaging instruction input from the operator. In step S204, the control apparatus 101 receives the image captured by the X-ray imaging apparatus 105. In step S205, the control apparatus 101 performs necessary image processing for the captured image.

In step S206, the control apparatus 101 determines whether imaging operation necessary for the examination is complete. Upon determining that necessary imaging operation is complete (YES in step S206), the control apparatus 101 terminates the examination. If the control apparatus 101 determines that necessary imaging operation is not complete (NO in step S206), the process returns to step S203.

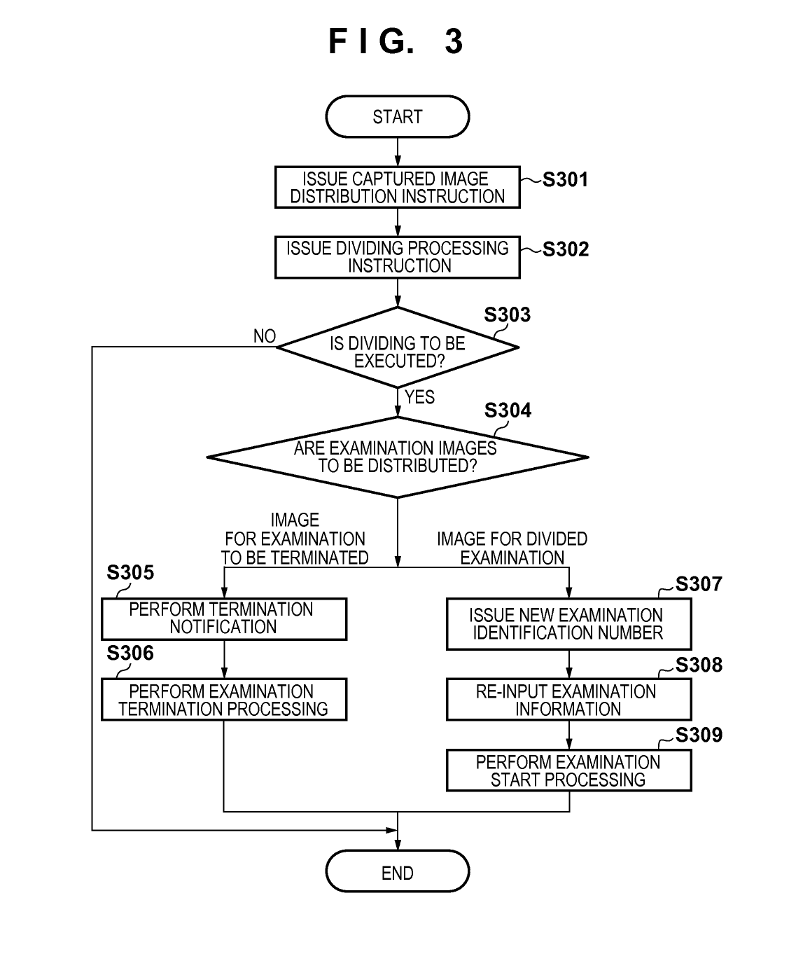

A procedure for examination dividing processing during an examination according to the first embodiment will be described next with reference to the flowchart of FIG. 3. If the operator finds out that he/she has mixed up between patients during the interval between the start of an examination and the end of the examination, he/she divides the examination under execution into the first examination associated with the mistaken patient and the second examination associated with the other patient by using the GUI displayed on the display unit 102 of the control apparatus 101 and the operation unit 103.

In step S301, the operation unit 103 receives an instruction to distribute captured images to discriminate images necessary for an examination under execution associated with the examination information input in step S201 and images necessary for an examination after dividing which is not associated with the examination information input in step S201. This processing is generally executed by making the operator select captured images, of the captured images displayed on the GUI, which should be included in the examination after dividing.

In step S302, the operation unit 103 receives a dividing processing instruction input from the operator. This processing is generally executed when the operator presses the examination dividing operation reception button displayed on the GUI.

In step S303, the display unit 102 prompts the operator to perform confirmation input for the execution of dividing operation in accordance with the dividing processing instruction input in step S302. This processing is generally executed by displaying a dialog for confirming whether to really execute examination dividing operation and making the operator perform confirmation input by selecting whether to really execute dividing operation. If the operator selects to execute examination dividing operation (YES in step S303), the process advances to step S304. If the operator does not select to execute examination dividing operation (NO in step S303), the control unit 104 terminates the processing. It is possible to reverse the execution order of the image distribution instruction reception processing in step S301, the dividing processing instruction reception processing in step S302, and the dialog display processing in step S303. That is, the control unit 104 may distribute images upon deciding the execution of examination dividing operation through dialog confirmation after the pressing of the examination dividing operation reception button.

In step S304, the control unit 104 distributes images necessary for the examination to be terminated (first examination) and the divided examination (second examination) based on the image distribution instruction issued in step S301. With this operation, the examination under execution includes only necessary images. In this captured image distribution processing, series numbers may be re-set for the captured images distributed to the respective examinations.

In step S305, the control unit 104 notifies the termination of the examination under execution. In step S306, the control unit 104 executes examination termination processing in accordance with an input from the operator.

In step S307, the control unit 104 newly creates an examination including the distributed and designated captured images in step S301 as images necessary for the examination after dividing, and issues a new examination identification number. This divides the examination under execution into the first examination (the examination under execution) associated with the input examination information and the second examination unassociated with the input examination information.

In step S308, the operation unit 103 receives examination information re-inputs such as patient information corresponding to a captured image with respect to the nearly created examination from the operator. This processing is executed when the operator inputs necessary patient information from the operation unit 103 such as a keyboard with respect to the dialog displayed on the GUI on the display unit 102. Alternatively, the apparatus may be configured to allow the operator to input information on a touch panel on the display unit 102. Examination information is re-input before the start of an examination after dividing.

In step S309, the control unit 104 executes examination start processing when the operator re-inputs examination information for the new examination. The examination after dividing is set in a state under execution and shifts to a state after the start of examination in step S202 in FIG. 2.

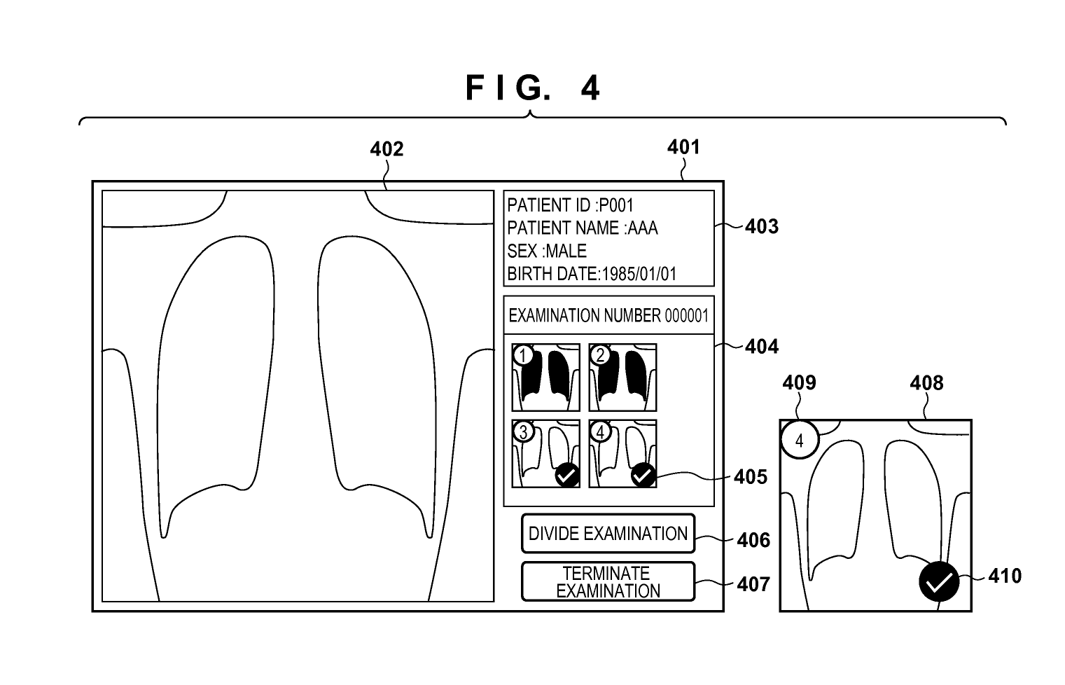

An example of the GUI displayed on the X-ray imaging system according to the first embodiment will be described next with reference to FIGS. 4 to 7. FIG. 4 shows an example of the GUI on the imaging screen. A window 401 is the GUI window displayed on the display unit 102 of the control apparatus 101 during an examination. The window 401 includes an acquired image display portion 402 which displays the captured image acquired from the X-ray imaging apparatus 105, a patient information display portion 403 which displays patient information corresponding to an examination under execution, and an examination information display portion 404 which displays an examination number, an imaging protocol, and thumbnail images 405 of captured images. The patient information displayed on the patient information display portion 403 includes a patient ID, patient name, sex, and birth date.

An image 408 is a detailed image of one of the thumbnail images 405. A number 409 is the image series number assigned to a captured image in an examination. The number 409 need not always be displayed on the thumbnail images 405 as long as the number is associated as information corresponding to the captured image. A selection icon 410 is an icon indicating the selected state of the captured image. A selected captured image is recognized as a processing target to be designated next. If imaging operation necessary for an examination is normally complete, the operator presses an examination termination button 407 to terminate the examination.

If the operator wants to divide some of the images displayed on the examination information display portion 404 for another examination during examination, he/she sets images, of the thumbnail images 405, which are to be divided for another examination in the selected state. This operation corresponds to the issuance of an instruction to distribute captured images (step S301). When the operator presses an examination dividing operation reception button 406 afterward, the control unit 104 issues a dividing processing instruction (step S302).

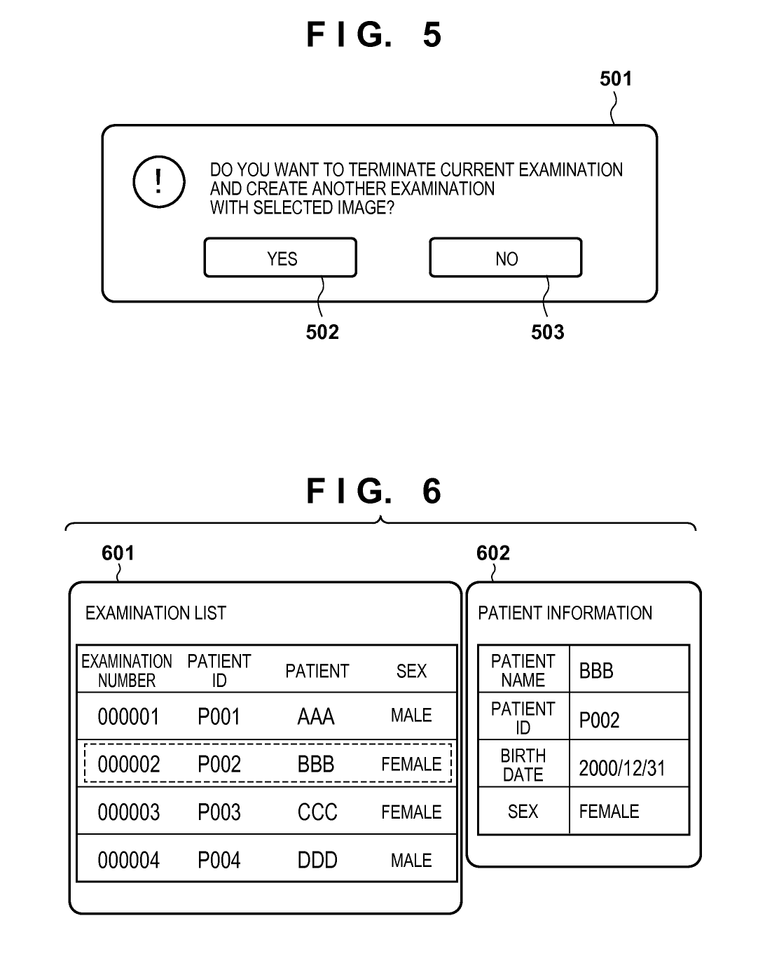

FIG. 5 shows an example of the dividing operation execution confirmation dialog displayed when an examination dividing processing instruction is issued. After the operator presses the examination dividing operation reception button 406, the control unit 104 displays a dividing operation execution confirmation dialog 501 (step S303). At this time, if the operator presses a decision button 502, the control unit 104 executes examination dividing processing. If the operator presses a stop button 503, the control unit 104 stops examination dividing processing.

Upon executing examination dividing, the control unit 104 distributes examination images (step S304). With regard to the images associated with the examination to be terminated (the first examination), the control unit 104 executes termination notification processing (step S305) and examination termination processing (step S306) in the examination under execution which is displayed on the window 401 while including the patient information displayed on the patient information display portion 403 and the images in the unselected state on the examination information display portion 404.

In contrast, the control unit 104 issues a new examination identification number for the image associated with divided examination (second examination) (step S307), and performs re-input of examination information necessary for newly created examination (step S308). FIG. 6 shows an example of a GUI for re-input of examination information for an examination after dividing. The control unit 104 displays a GUI for re-input of examination information suitable for an operation form. In an operation form of inputting examination information from an external system by using MWL (Modality Worklist) or the like, the control unit 104 displays an examination list dialog 601. In contrast, in an operation form of manually inputting patient information for each examination, the control unit 104 displays a manual input dialog 702. When examining an urgent patient, the control apparatus 101 automatically creates and issues patient information. After re-input of examination information, the control unit 104 performs examination start processing (step S309) and starts an examination after dividing.

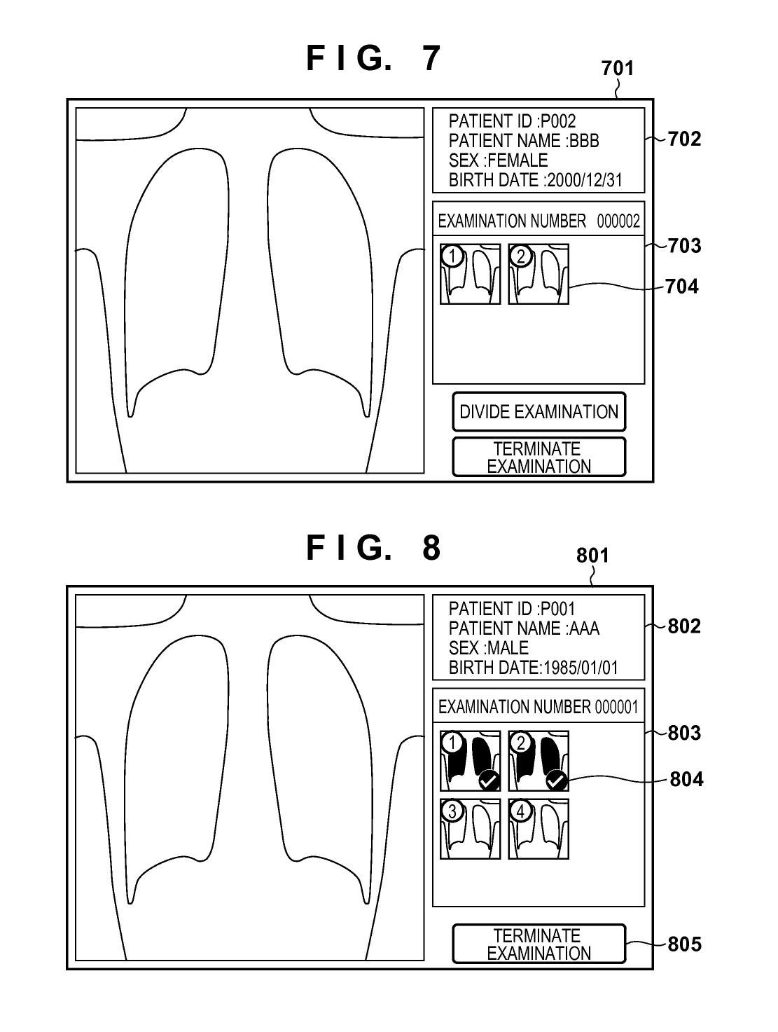

FIG. 7 shows an example of an imaging screen GUI after dividing according to the first embodiment. On a window 701 displayed during an examination, the image selected on the examination information display portion 404 in FIG. 4 is held as a thumbnail image 704 of a captured image, and the information input by examination information re-input processing (step S308) is set on the patient information display portion 702 and an examination information display portion 703. If the examination list dialog 601 in FIG. 6 indicates that the examination number is 000002, the patient ID is P002, the patient name is BBB, and the sex is female, these pieces of information are displayed on the patient information display portion 702 and the examination information display portion 703. With regard to this examination, if there is an imaging operation unexecuted in a series of examination procedures, it is possible to execute the imaging operation.

As described above, according to this embodiment, it is possible to easily divide some of images in an examination under execution for another examination. This can reduce the number of times of operation performed by the operator, improve the operator's operability, shorten the wait time for a patient upon occurrence of mix-up, and improve the throughput and accuracy of examinations.

### Second Embodiment

The first embodiment has exemplified the arrangement in which the operator performs GUI operation to issue a captured image distribution instruction (S301) to make images to be included in an examination after dividing selectable when performing dividing processing, and presses an examination dividing operation reception button 406 to issue an instruction to perform dividing processing (step S302). In contrast to this, the second embodiment will exemplify an arrangement in which the operator sets images which he/she does not want to include in an examination after dividing in the selected state.

FIG. 8 shows an example of an imaging screen GUI after dividing according to the second embodiment. A window 801 is a GUI window displayed on a display unit 102 of a control apparatus 101 during an examination.

The window 801 includes a patient information display portion 802 which displays patient information corresponding to an examination under execution and an examination information display portion 803 which displays an examination number, an imaging protocol, thumbnail images 804 of captured images. The patient information displayed on the patient information display portion 802 includes a patient ID, patient name, sex, and birth date. When imaging required for an examination is normally completed, the apparatus terminates the examination when the operator presses an examination termination button 805.

As shown in FIG. 8, if the operator wants to divide some of the images displayed on the examination information display portion 803 for another examination, the apparatus sets captured images, of the thumbnail images 804, which correspond to the examination under execution in the selected state. That is, unlike the first embodiment, the second embodiment sets, as images to be set in the selected state for examination dividing operation, images which the operator does not want to divide instead of images which he/she wants to divide for another examination. This operation corresponds to the issuance of a captured image distribution instruction (step S301). When the operator presses the examination termination button 805 afterward, the apparatus issues a dividing processing instruction (step S302). Since other processing is the same as that in the first embodiment, a description of the processing will be omitted.

If captured images for an examination are arranged in chronological order, images included in the examination under execution are probably arranged before images captured by imaging operation after the operator has mixed up between patients during the examination. It is therefore possible to issue a captured image distribution instruction (step S301) by designating a boundary position between images to be included in an examination under execution and images to be included in an examination after dividing. Assume that when there are two captured images, the operator presses the examination dividing operation reception button 406 without issuing a captured image distribution instruction (step S301), thereby issuing a dividing processing instruction (step S302). In this case, the apparatus may perform examination dividing processing while including one previously captured image in an examination under execution.

### Third Embodiment

A plurality of patients are sometimes imaged in one examination at the time of execution of a health check or the like. This occurs when the operator forgets to perform examination termination processing when imaging the same region of each person in succession. The operator should capture only one image of the same examination region to be included in one examination.

The third embodiment will exemplify an arrangement configured to notify the operator when he/she has consecutively imaged the same examination region a plurality of times and to display a dialog for making the operator confirm the necessity of dividing operation.

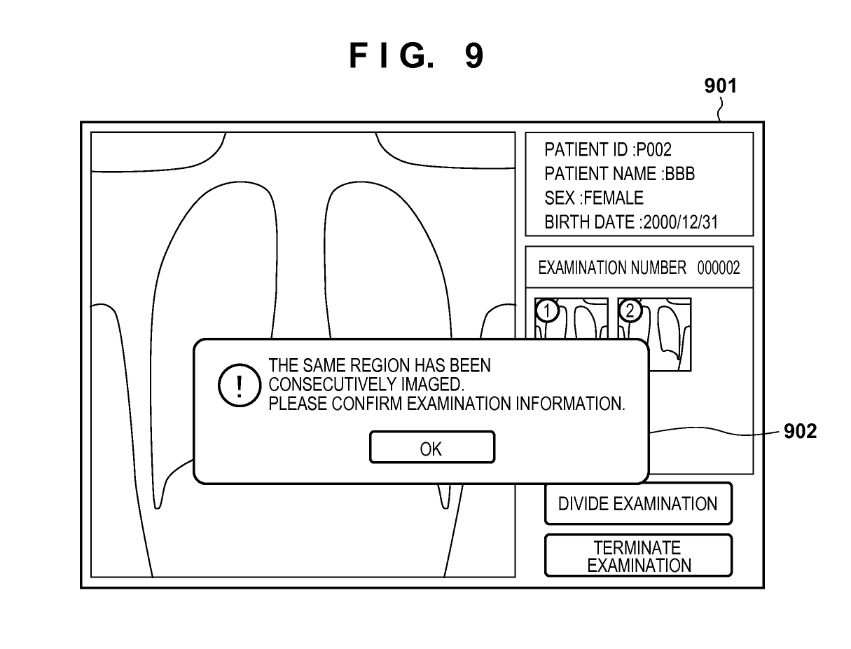

FIG. 9 shows an example of the warning dialog displayed when the same region has been consecutively imaged according to the third embodiment. A window 901 is a GUI window displayed on a display unit 102 of a control apparatus 101 during an examination. When the same region has been consecutively imaged, the control apparatus 101 notifies the operator of the corresponding information by displaying a dialog 902 for prompting the operator to confirm examination information such as patient information.

After closing the dialog 902, the operator confirms examination information, and performs examination dividing processing if necessary. This apparatus may be configured not to display any warning dialog at the occurrence of misphotographing or at the time of re-imaging operation.

As has been described above, this embodiment makes the operator easily notice that he/she has forgotten the end of an examination and imaged the same region of a patient more than once. This improves the accuracy of the examination.

Note that this apparatus may perform display control to make the display unit display a display screen for the execution of the first examination in accordance with an instruction to start the first examination, store information of the started examination in association with the radiographic images obtained by imaging, and display, in the display screen for the execution of the first examination, a GUI for receiving the operation of associating, instead of the information of the first examination, information of the second examination different from the information of the first examination, with respect to at least one of a plurality of radiographic images with which the information of the first examination is associated. This makes it possible to manage a plurality of radiographic images in association with each examination.

The present invention can improve the operator's operability and reduce errors because it is possible to decrease the number of operation steps necessary for information correction when the operator notices that he/she has mixed up between patients during an examination. In addition, improving the operability can shorten the wait time generated accompanying correction of information of a patient under examination.

### Other Embodiments

Aspects of the present invention can also be realized by a computer of a system or apparatus (or devices such as a CPU or MPU) that reads out and executes a program recorded on a memory device to perform the functions of the above-described embodiment(s), and by a method, the steps of which are performed by a computer of a system or apparatus by, for example, reading out and executing a program recorded on a memory device to perform the functions of the above-described embodiment(s). For this purpose, the program is provided to the computer for example via a network or from a recording medium of various types serving as the memory device (for example, computer-readable storage medium).

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-165138 filed on Jul. 25, 2012, which is hereby incorporated by reference herein in its entirety.

What is claimed is:

1. An X-ray imaging apparatus comprising:
an examination information input unit configured to input examination information necessary for an examination;
an examination dividing operation reception unit configured to receive an instruction to divide the examination into a first examination associated with the examination information and a second examination unassociated with the examination information, during an interval between a start of the examination and an end of the examination; and
an image distribution instruction reception unit configured to receive an instruction to distribute images which have been captured in the examination in association with one of the first examination and the second examination.

2. The apparatus according to claim 1, wherein said examination information input unit inputs examination information necessary for the second examination when the examination is divided.

3. The apparatus according to claim 2, wherein said examination information input unit inputs examination information necessary for the second examination before a start of the second examination.

4. The apparatus according to claim 1, further comprising an assigning unit configured to assign the second examination with an examination identification number different from an examination identification number of the first examination.

5. The apparatus according to claim 1, further comprising:
a start instruction reception unit configured to receive an instruction to start the examination; and
a termination instruction reception unit configured to receive an instruction to terminate the examination.

6. The apparatus according to claim 5, wherein said termination instruction reception unit receives an instruction to terminate the first examination when the examination is divided.

7. The apparatus according to claim 1, wherein said examination information input unit inputs the examination information upon receiving an input from an operator,
inputs examination information selected by the operator from a plurality of pieces of examination information acquired from an external device, or
automatically creates and inputs the examination information.

8. The apparatus according to claim 1, wherein said image distribution instruction reception unit distributes the captured images by receiving selection of an image to be associated with the first examination,
receiving selection of an image to be associated with the second examination, or
receiving designation of a boundary position between an image to be associated with the first examination and an image to be associated with the second examination.

9. The apparatus according to claim 1, further comprising a setting unit configured to set a series number of an image distributed in association with the second examination and a series number of an image distributed in association with the second examination.

10. The apparatus according to claim 1, further comprising a notification unit configured to notify that the same examination region has been imaged a plurality of times in an examination according to one piece of examination information.

11. The apparatus according to claim 10, further comprising a confirmation unit configured to confirm, upon reception of an instruction to divide an examination from said examination dividing operation reception unit, whether to divide the examination.

12. An X-ray imaging apparatus configured to manage a plurality of radiographic images in association with each examination, the apparatus comprising:
a display control unit configured to cause a display unit to display a display screen for execution of the first examination in accordance with an instruction to start the first examination; and
a storage unit configured to store information of the started examination in association with a radiographic image obtained by imaging,
wherein said display control unit further causes the display unit to display, in the display screen for execution of the first examination, a GUI for receiving operation of associating, instead of the information of the first examination, information of a second examination which is different from the information of the first examination with at least one of a plurality of radiographic images with which the information of the first examination is associated.

13. A method of controlling an X-ray imaging apparatus, the method comprising:
an examination information input step of inputting examination information necessary for an examination;
an examination dividing operation reception step of receiving an instruction to divide the examination into a first examination associated with the examination information and a second examination unassociated with the examination information, during an interval between a start of the examination and an end of the examination; and
an image distribution instruction reception step of receiving an instruction to distribute images which have been captured in the examination in association with one of the first examination and the second examination.

14. A non-transitory computer-readable storage medium storing a computer program for causing a computer to execute each step of a method of controlling an X-ray imaging apparatus defined in claim 13.
